(19) United States
(12) Patent Application Publication
(10) Pub. No.: US 2021/0100511 A1
(43) Pub. Date: Apr. 8, 2021

(54) LIVING BODY-ATTACHABLE ELECTRODE AND METHOD OF MANUFACTURING LIVING BODY-ATTACHABLE ELECTRODE

(71) Applicant: Murata Manufacturing Co., Ltd., Nagaokakyo-shi (JP)

(72) Inventor: Kazuki Iwasaki, Nagaokakyo-shi (JP)

(21) Appl. No.: 17/125,394

(22) Filed: Dec. 17, 2020

Related U.S. Application Data
(63) Continuation of application No. PCT/JP2019/015778, filed on Apr. 11, 2019.

(30) Foreign Application Priority Data
Jun. 29, 2018 (JP) 2018-125390

(51) Int. Cl. A61B 5/00; H01B 5/14

(52) U.S. Cl. CPC A61B 5/6833; A61B 2562/164; A61B 2562/125; H01B 5/14

(57) ABSTRACT

A living body-attachable electrode is provided with a first stretchable substrate. A conductive pattern may be provided on one main surface of the first stretchable substrate and including an electrode part and a wiring part connected to the electrode part. A second stretchable substrate may be provided on the one main surface of the first stretchable substrate to cover at least the wiring part of the conductive pattern and having an opening exposing at least a part of the electrode part. A gel electrode may be provided in the opening of the second stretchable substrate and conductive to the electrode part, in which the gel electrode is provided on a main surface of the second stretchable substrate to spread outward from the opening of the second stretchable substrate. The main surface may not be in contact with the first stretchable substrate.

### CROSS REFERENCE TO RELATED APPLICATIONS

This application is a continuation of PCT Application No. PCT/JP2019/015778, filed Apr. 11, 2019, which claims priority to Japanese Patent Application No. 2018-125390, filed Jun. 29, 2018, the entire contents of each of which are hereby incorporated in their entirety.

### TECHNICAL FIELD

Aspects of the present disclosure are directed to a living body-attachable electrode and a method of manufacturing a living body-attachable electrode.

### BACKGROUND OF THE INVENTION

In recent years, a state of a human body and the like has been managed by acquiring and analyzing biometric information.

For example, Japanese Patent Application Laid-Open No. 2017-143257 (“JP '257”), discloses a living body-attachable electrode in which an electrode part and a wiring part are formed on a stretchable substrate and biometric information is acquired through a biogel provided on a surface of the electrode part.

In the living body-attachable electrode as disclosed in JP '257, when the stretchable substrate is stretched, the biogel (hereinafter referred to as gel electrode) may be laterally displaced. In that case, the electrode part or the wiring part formed on the stretchable substrate may be exposed and contact a living body. The electrode part and the wiring part, for which not biocompatible materials are often used, desirably do not contact the living body as much as possible.

The present invention has been made to solve the above problems. An object of the present disclosure is to provide a living body-attachable electrode capable of preventing an electrode part or a wiring part from being exposed upon lateral displacement of a gel electrode and avoiding contact with a living body. Another object of the present disclosure is to provide a method of manufacturing the living body-attachable electrode.

### SUMMARY OF THE INVENTION

Accordingly, it is an object of the present disclosure to provide a living body-attachable electrode which includes a first stretchable substrate, a conductive pattern provided on one main surface of the first stretchable substrate and including an electrode part and a wiring part connected to the electrode part, a second stretchable substrate provided on the one main surface of the first stretchable substrate to cover at least the wiring part of the conductive pattern and having an opening exposing at least a part of the electrode part, and a gel electrode provided in the opening of the second stretchable substrate and conductive to the electrode part, in which the gel electrode is provided on a main surface of the second stretchable substrate to spread outward from the opening of the second stretchable substrate, the main surface being not in contact with the first stretchable substrate.

Accordingly, it is an object of the present disclosure to provide a method of manufacturing a living body-attachable electrode which includes forming a conductive pattern including an electrode part and a wiring part connected to the electrode part on one main surface of the first stretchable substrate, placing a second stretchable substrate having an opening on the one main surface of the first stretchable substrate, covering at least the wiring part of the conductive pattern with the second stretchable substrate, and exposing at least a part of the electrode part from the opening of the second stretchable substrate, applying a gel electrode material inside the opening of the second stretchable substrate, and forming a gel electrode conductive to the electrode part by curing the gel electrode material.

According to the present disclosure the living body-attachable electrode may be capable of preventing the electrode part or the wiring part from being exposed upon lateral displacement of the gel electrode and avoiding contact with the living body.

Additional advantages and novel features of the system of the present disclosure will be set forth in part in the description that follows, and in part will become more apparent to those skilled in the art upon examination of the following or upon learning by practice of the disclosure.

### BRIEF DESCRIPTION OF THE DRAWINGS

In the descriptions that follow, like parts are marked throughout the specification and drawings with the same numerals, respectively. The drawings are not necessarily drawn to scale and certain drawings may be shown in exaggerated or generalized form in the interest of clarity and conciseness. The disclosure itself, however, as well as a mode of use, further features and advances thereof, will be understood by reference to the following detailed description of illustrative implementations of the disclosure when read in conjunction with reference to the accompanying drawings, wherein:

FIG. 1 is a sectional view schematically showing a living body-attachable electrode in accordance with aspects of the present disclosure;

FIG. 2 is a top view schematically showing the living body-attachable electrode in accordance with aspects of the present disclosure;

FIG. 3 is a sectional view schematically showing an example of a state in which the living body-attachable electrode shown in FIG. 1 is attached to a human body in accordance with aspects of the present disclosure;

FIGS. 4 (A)-4(B) are sectional views schematically showing an example of a state in which a gel electrode extending to the outside of an opening of a second stretchable substrate is laterally displaced in accordance with aspects of the present disclosure;

FIGS. 5 (A)-5(B) are sectional views schematically showing an example of a state in which the gel electrode not extending to the outside of the opening of the second stretchable substrate is laterally displaced in accordance with aspects of the present disclosure;

FIG. 6 is a sectional view schematically showing a living body-attachable electrode in accordance with aspects of the present disclosure;

FIG. 7 is a sectional view showing a relationship between a radius of a through hole of an electrode part and a thickness of the electrode part in accordance with aspects of the present disclosure;

FIGS. 8 (A)-8(C) are distribution maps of shear force on an upper surface of the electrode part obtained by simulation in accordance with aspects of the present disclosure;

FIG. 9 is a sectional view schematically showing a living body-attachable electrode in accordance with aspects of the present disclosure;

FIG. 10 is a sectional view schematically showing a living body-attachable electrode in accordance with aspects of the present disclosure;

FIG. 11 is a sectional view schematically showing a living body-attachable electrode in accordance with aspects of the present disclosure;

FIGS. 12 (A)-12(E) are sectional views schematically showing a method of manufacturing the living body-attachable electrode in accordance with aspects of the present disclosure; and

FIGS. 13 (A)-13(E) are top views schematically showing the method of manufacturing the living body-attachable electrode in accordance with aspects of the present disclosure.

### DETAILED DESCRIPTION

Hereinafter, a living body-attachable electrode of the present disclosure will be described.

However, the present disclosure is not limited to the following configurations, and can be appropriately modified and applied without changing the gist of the present disclosure. Note that a combination of two or more of individual desirable configurations of the present disclosure described below is also the present invention.

Aspects of the disclosure described below are mere examples, and the configurations can be partially replaced or combined in the different aspects of the disclosure. The description of items common to different aspects will be omitted, and different points will be described. In particular, the same effects by the same configuration will not be sequentially referred to for each aspect.

In the following description, unless each aspect is distinguished, the present invention is simply referred to as a “living body-attachable electrode of the present disclosure.”

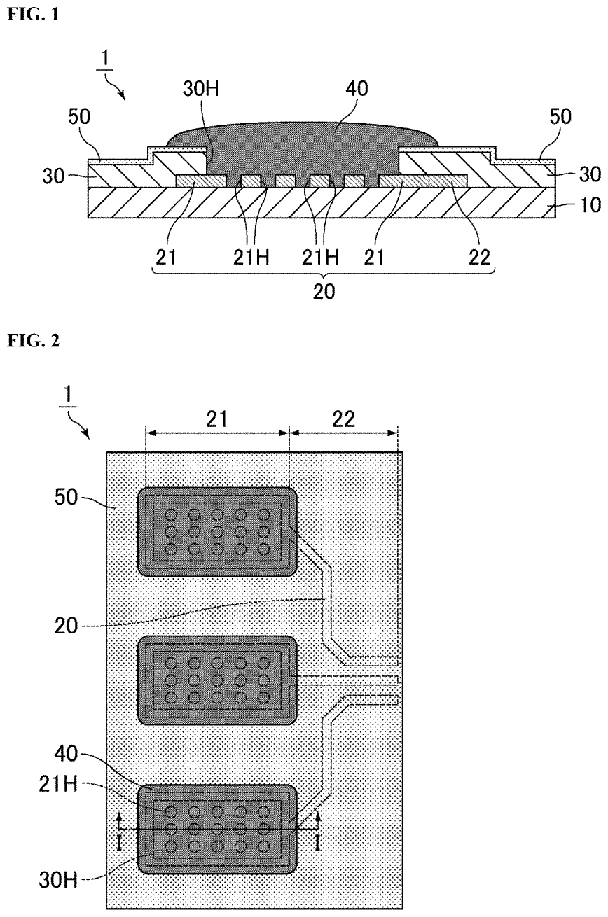

FIG. 1 is a sectional view schematically showing a living body-attachable electrode in accordance with aspects of the present disclosure. FIG. 2 is a top view schematically showing the living body-attachable electrode in accordance with aspects of the present disclosure. Note that FIG. 1 is a sectional view taken along line I-I of the living body-attachable electrode shown in FIG. 2. In FIG. 2, a conductive pattern is transparently shown.

A thickness of each part shown in FIG. 1 has been changed as appropriate to clarify and simplify the drawing, and does not represent a relationship between the actual thicknesses. The same applies to other sectional views.

A living body-attachable electrode 1 shown in FIGS. 1 and 2 includes a first stretchable substrate 10, a conductive pattern 20, a second stretchable substrate 30, and a gel electrode 40. As shown in FIGS. 1 and 2, the living body-attachable electrode 1 desirably further includes an adhesive layer 50 on a main surface of the second stretchable substrate 30, the main surface being not in contact with the first stretchable substrate 10.

The conductive pattern 20 is provided on one main surface of the first stretchable substrate 10, and includes an electrode part 21 and a wiring part 22 connected to the electrode part 21. In the living body-attachable electrode 1, the electrode part 21 has a plurality of through holes 21H penetrating in a thickness direction (vertical direction in FIG. 1). Instead of the through holes 21H, the electrode part 21 may have a plurality of recesses recessed in the thickness direction from the main surface not in contact with the first stretchable substrate 10.

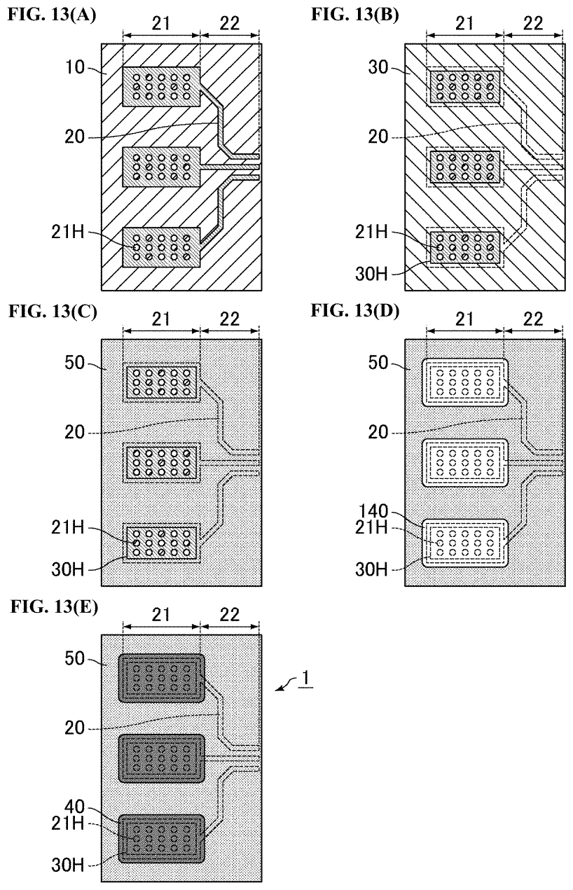

The second stretchable substrate 30 is provided on the one main surface of the first stretchable substrate 10 so as to cover at least the wiring part 22 of the conductive pattern 20, and has an opening 30H exposing at least a part of the electrode part 21. In the living body-attachable electrode 1, in addition to the wiring part 22, an entire periphery of the electrode part 21 is covered with the second stretchable substrate 30 (see FIG. 13B).

The gel electrode 40 is provided in the opening 30H of the second stretchable substrate 30 and is conductive to the electrode part 21. In the living body-attachable electrode 1, the gel electrode 40 also enters into the through holes 21H of the electrode part 21.

Further, the gel electrode 40 is also provided on the main surface of the second stretchable substrate 30 not in contact with the first stretchable substrate 10 so as to spread outward from the opening 30H of the second stretchable substrate 30.

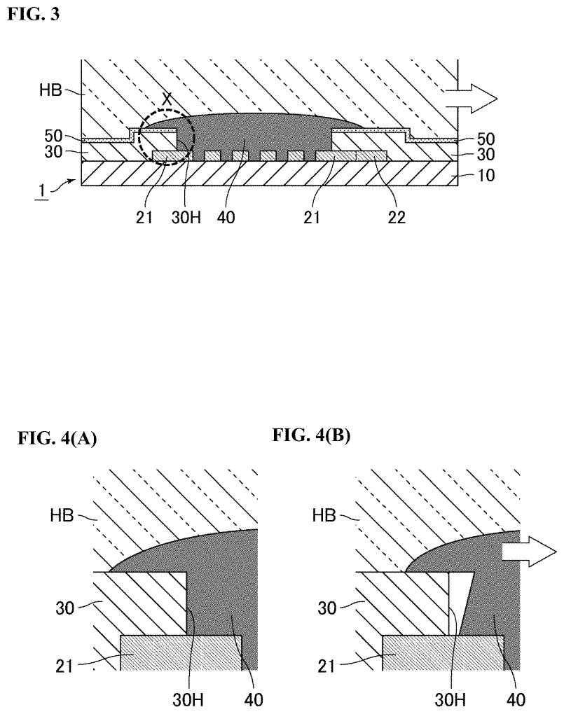

FIG. 3 is a sectional view schematically showing an example of a state in which the living body-attachable electrode shown in FIG. 1 is attached to a human body.

As shown in FIG. 3, the living body-attachable electrode 1 can measure a biological signal through the gel electrode 40 with the gel electrode 40 attached to a living body such as a human body.

FIGS. 4A and 4B are sectional views schematically showing an example of a state in which the gel electrode extending to the outside of the opening of the second stretchable substrate is laterally displaced. FIG. 4A is an enlarged view of an X region in FIG. 3. In FIGS. 4A and 4B, the adhesive layer 50 is omitted.

For example, when the first stretchable substrate 10 and the second stretchable substrate 30 are stretched as a human body HB moves in a direction of an arrow shown in FIG. 3, the gel electrode 40 may be laterally displaced in a direction of an arrow shown in FIG. 4B. Even when the gel electrode 40 is laterally displaced in this way, causing a gap in a direction of the main surface between the second stretchable substrate 30 and the gel electrode 40, the electrode part 21 is not exposed as shown in FIG. 4B if the gel electrode 40 extends to the outside of the opening 30H of the second stretchable substrate 30 as shown in FIG. 4A, thereby avoiding a contact between the human body HB and the electrode part 21.

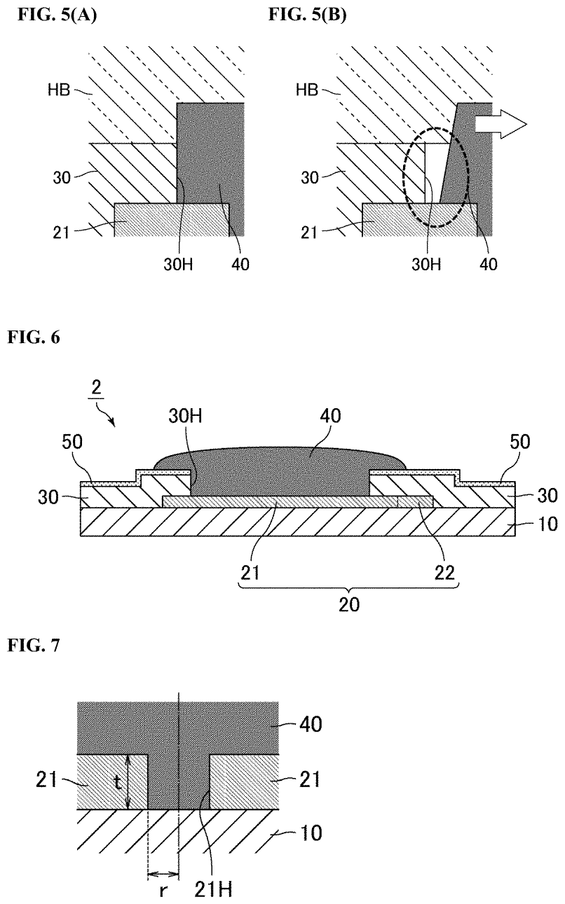

FIGS. 5A and 5B are sectional views schematically showing an example of a state in which the gel electrode not extending to the outside of the opening of the second stretchable substrate is laterally displaced. If the gel electrode 40 does not extend to the outside of the opening 30H of the second stretchable substrate 30 as shown in FIG. 5A, a gap is formed between the second stretchable substrate 30 and the gel electrode 40 in a direction of the main surface, and the electrode part 21 is exposed as indicated by an encircling broken line when the gel electrode 40 is laterally displaced in a direction of an arrow shown in FIG. 5B. An actual distance from the human body HB to the electrode part 21 is significantly short, and the skin of the human body HB may enter the gap between the second stretchable substrate 30 and the gel electrode 40 and may contact the electrode part 21. Further, water discharged from the human body HB may cause components of the electrode part 21 to elute and contact the human body HB.

In the living body-attachable electrode according to an aspect of the disclosure, as described above, the gel electrode extends to the outside of the opening of the second stretchable substrate, and thus the electrode part or the wiring part is prevented from being exposed in response to the lateral displacement of the gel electrode, thereby avoiding a contact with a living body such as a human body.

Further, in the living body-attachable electrode according to an aspect of the disclosure, the second stretchable substrate is stacked on the first stretchable substrate, which can seal the wiring part while having a stretchable function.

In the living body-attachable electrode according to an aspect of the disclosure, the first stretchable substrate and the second stretchable substrate include, for example, a stretchable resin material. Examples of the resin material configuring the first stretchable substrate and the second stretchable substrate include thermoplastic polyurethane and the like. The material configuring the first stretchable substrate may be identical to or different from the material configuring the second stretchable substrate.

In the living body-attachable electrode according to an aspect of the disclosure, thicknesses of the first stretchable substrate and of the second stretchable substrate are not limited. However, in order not to inhibit the stretchability of a surface of the living body when attached to the living body, the first stretchable substrate and the second stretchable substrate desirably have a thickness of 100 μm or less, and more desirably have a thickness of 1 μm or less. Further, the thicknesses of the first stretchable substrate and the second stretchable substrate are desirably 0.1 μm or more. The thickness of the first stretchable substrate may be identical to or different from the thickness of the second stretchable substrate.

In the living body-attachable electrode according to an aspect of the disclosure, the electrode part and the wiring part configuring the conductive pattern include a conductive material. Examples of the conductive material configuring the electrode part and the wiring part include a mixture of metal particles such as silver and copper and an elastomer-based resin such as silicone. The conductive material configuring the electrode part may be identical to or different from the conductive material configuring the wiring part.

In the living body-attachable electrode according to an aspect of the disclosure, thicknesses of the electrode part and the wiring part configuring the conductive pattern are not limited, but are desirably 100 μm or less, and more desirably 50 μm or less. Further, the thicknesses of the electrode part and the wiring part are desirably 1 μm or more. The thickness of the electrode part may be identical to or different from the thickness of the wiring part. The first stretchable substrate and the second stretchable substrate may be thinner or thicker than the electrode part and the wiring part, but is desirably thinner than both the electrode part and the wiring part, which can improve the stretchability of the living body-attachable electrode.

In the living body-attachable electrode according to an aspect of the disclosure, the gel electrode includes a conductive gel material containing, for example, water, alcohol, a moisturizer, an electrolyte, and the like. Examples of such a gel material include hydrogel and the like.

As will be described below, the gel electrode is desirably formed by applying a gel electrode material inside the opening of the second stretchable substrate and curing the gel electrode material. Unlike a method of forming the gel electrode by placing a gel sheet having a predetermined shape in the opening, in a method of applying the gel electrode material inside the opening and curing the gel electrode material, a core material for maintaining the shape of the gel electrode does not have to be used. Such a core material includes a non-woven fabric or a woven fabric. Examples of the material configuring the non-woven fabric or woven fabric include natural fibers such as cellulose, silk, and linen, synthetic fibers such as polyester, nylon, rayon, polyethylene, polypropylene, and polyurethane, and blends thereof.

For the above reasons, in the living body-attachable electrode according to an aspect of the disclosure, a content of the core material in the gel electrode is desirably 50% by weight or less, more desirably 10% by weight or less, and particularly desirably below or equal to a detection limit.

In the present disclosure, the content of the core material in the gel electrode is a value measured by using a microbalance or the like.

In the living body-attachable electrode according to an aspect of the disclosure, a length of the gel electrode protruding from the opening of the second stretchable substrate onto the main surface of the second stretchable substrate is not limited, but is desirably 1 μm or more, and more desirably 1 mm or more.

Further, the length of the gel electrode protruding from the opening of the second stretchable substrate onto the main surface of the second stretchable substrate is desirably 50 mm or less.

In the living body-attachable electrode according to an aspect of the disclosure, the gel electrode provided outside of the opening of the second stretchable substrate protrudes from the second stretchable substrate in the thickness direction. Meanwhile, the gel electrode provided in the opening of the second stretchable substrate does not have to protrude from the second stretchable in the thickness direction, but desirably protrudes from the second stretchable substrate in the thickness direction as shown in FIG. 1. In particular, as shown in FIG. 1, the gel electrode desirably has a protruding shape being thicker from a peripheral edge toward a center.

The gel electrode, protruding from the second stretchable substrate, can follow unevenness of the living body with the gel electrode attached to the living body, thereby improving adhesion to the gel electrode. Consequently, this can reduce an interface resistance between the gel electrode and the living body.

In particular, the gel electrode having the protruding shape protruding causes a gap between a peripheral edge and the human body. Thus, a spread of the gel electrode pressed against the human body by the attachment moves to the gap. Then, the gel electrode can be prevented from spreading in a direction of a plane as compared with a flat gel electrode protruding. As a result, this can prevent short-circuiting between adjacent gel electrodes. Furthermore, an amount of gel electrode used can be reduced, and a material cost can be suppressed.

In the living body-attachable electrode according to an aspect of the disclosure, a height of the gel electrode protruding from the second stretchable substrate in the thickness direction is not limited, but is desirably 1 μm or more, and more desirably 100 μm or more. Further, the height of the gel electrode protruding from the second stretchable substrate in the thickness direction is desirably 1 mm or less.

FIG. 6 is a sectional view schematically showing a living body-attachable electrode according to an aspect of the disclosure.

A living body-attachable electrode 2 shown in FIG. 6 has the same configuration as the living body-attachable electrode 1 shown in FIG. 1, except that the electrode part 21 does not have the through holes 21H.

In the living body-attachable electrode according to an aspect of the disclosure, as shown in FIG. 6, the electrode part does not have to have the through holes penetrating the electrode part in the thickness direction, but desirably has a plurality of recesses that are recessed in the thickness direction from the main surface not in contact with the first stretchable substrate. As shown in FIGS. 1 and 2, the recesses are desirably through holes that penetrate the electrode part in the thickness direction.

In this case, the gel electrode enters the recesses such as the through holes, and the gel electrode is less likely to be displaced from the electrode part due to an anchor effect. This can relax shear force between the gel electrode and the electrode part caused by stretching on the living body upon attachment of the gel electrode to the living body.

FIG. 7 is a sectional view showing a relationship between a radius of the through holes of the electrode part and the thickness of the electrode part.

When the radius of each through hole 21H of the electrode part 21 is r and the thickness of the electrode part 21 is t, an area where the gel electrode 40 contacts the electrode part 21 decreases by πr² as a bottom area of each through hole 21H but increases by 2πrt as a side area of each through hole 21H as compared with a state without the through holes 21H. Therefore, when 0≤2πrt−πr², that is, r≤2t, the shear force between the gel electrode 40 and the electrode part 21 can be relaxed without decreasing the area where the gel electrode 40 contacts the electrode part 21 as compared with the state without the through holes 21H.

For the above reasons, in the living body-attachable electrode according to an aspect of the disclosure, r≤2t is desirably satisfied when the radius of the through holes of the electrode part is r and the thickness of the electrode part is t.

One exemplary aspect of the disclosure includes by using a simulation, the shear force between the gel electrode 40 and the electrode part 21 generated by the movement of the human body HB in the direction of the arrow shown in FIG. 3.

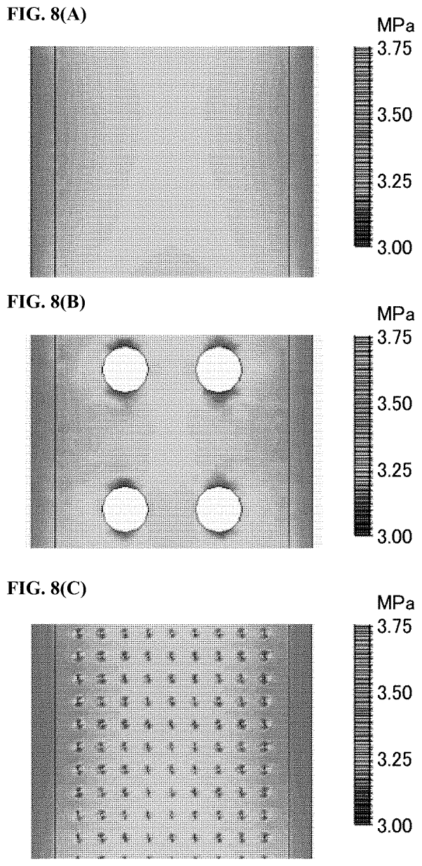

FIGS. 8 (A)-(C) are distribution maps of the shear force on an upper surface of the electrode part obtained by the simulation. FIG. 8A shows the distribution of the shear force when the electrode part has no through holes, and FIGS. 8B and 8C show the distribution of the shear force when the electrode part has the through holes. In FIGS. 8A, 8B, and 8C, the thickness t of the electrode part is 15 μm.

Compared with FIG. 8A in which the electrode part has no through holes, the shear force is generally reduced in FIG. 8B in which the electrode part has the through holes. In particular, there is a region where the shear force is intensively reduced near the through holes. It is therefore considered that the shear force between the gel electrode and the electrode part can be relaxed by providing the through holes in the electrode part.

In FIG. 8C, the radius r of the through holes in the electrode part is 30 μm. The thickness t of the electrode part is 15 μm, and thus r=2t. The number of through holes is 9×27=243.

Also, in FIG. 8C, because the shear force is intensively reduced in near the through holes, and the through holes are entirely arranged, the shear force is relaxed in the entire electrode part. It is therefore considered that, by satisfying r≤2t, the shear force between the gel electrode and the electrode part can be relaxed without reducing the area where the gel electrode contacts the electrode part as compared with the state without the through holes.

In the living body-attachable electrode according to an aspects of the disclosure, when the electrode part has the plurality of recesses, the shapes of the recesses may all be identical, or at least a part of the shapes may be different. Further, the electrode part may have both the recesses and the through holes.

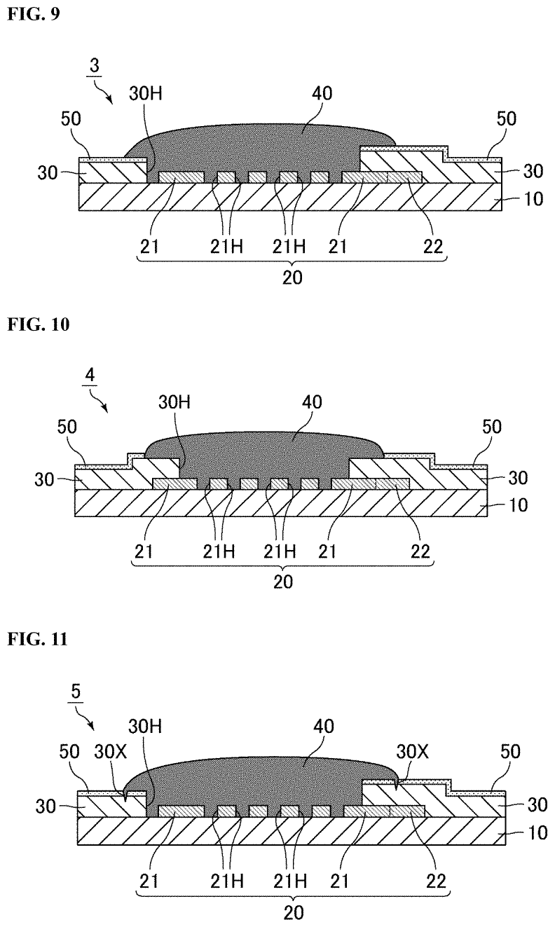

FIG. 9 is a sectional view schematically showing a living body-attachable electrode according to an aspect of the disclosure.

A living body-attachable electrode 3 shown in FIG. 9 has the same configuration as the living body-attachable electrode 1 shown in FIG. 1 except that a part around the electrode part 21 that is unconnected to the wiring part 22 is not covered with the second stretchable substrate 30. In the living body-attachable electrode 1 shown in FIG. 1 and in the living body-attachable electrode 2 shown in FIG. 6, the part around the electrode part 21 unconnected to the wiring part 22 does not have to be covered with the second stretchable substrate 30.

In the living body-attachable electrode according to an aspect of the disclosure, it is sufficient that at least the wiring part of the conductive pattern is covered with the second stretchable substrate. As shown in FIG. 1, the entire periphery of the electrode part may be covered with the second stretchable substrate. Alternatively, the part around the electrode part unconnected to the wiring part may not have to be covered with the second stretchable substrate as shown in FIG. 9.

In the living body-attachable electrode according to an aspect of the disclosure, it is sufficient that at least a part of the electrode part is exposed from the opening of the second stretchable substrate. Thus, as shown in FIG. 1, a part of the electrode part may be exposed from the opening of the second stretchable substrate, or the entire electrode part may be exposed from the opening of the second stretchable substrate. When the entire electrode part is exposed from the opening of the second stretchable substrate, the wiring part of the conductive pattern may be covered with the second stretchable substrate, and the electrode part is not covered with the second stretchable substrate.

As shown in FIG. 1, the living body-attachable electrode according to an aspect of the disclosure may desirably further include an adhesive layer on the main surface of the second stretchable substrate, the main surface being not in contact with the first stretchable substrate.

The adhesive layer provided on the main surface of the second stretchable substrate can improve adhesiveness to the living body.

FIG. 10 is a sectional view schematically showing a living body-attachable electrode according to an aspect of the disclosure.

A living body-attachable electrode 4 shown in FIG. 10 has the same configuration as the living body-attachable electrode 1 shown in FIG. 1 except that the adhesive layer 50 is not provided between the main surface of the second stretchable substrate 30 and the gel electrode 40, the main surface being not in contact with the first stretchable substrate 10. In the living body-attachable electrode 1 shown in FIG. 1, the living body-attachable electrode 2 shown in FIG. 6 and the living body-attachable electrode 3 shown in FIG. 9, the adhesive layer 50 may not have to be provided between the main surface of the second stretchable substrate 30 and the gel electrode 40, the main surface being not in contact with the first stretchable substrate 10.

The adhesive layer, which is included in the living body-attachable electrode according to an aspect of the disclosure, does not have to be provided between the main surface of the second stretchable substrate not in contact with the first stretchable substrate and the gel electrode as shown in FIG. 10, but is preferably provided between the main surface of the second stretchable substrate not in contact with the first stretchable substrate and the gel electrode as shown in FIG. 1.

In this case, the adhesive layer is less likely to peel off from the second stretchable substrate, and the gel electrode is less likely to be laterally displaced. It is therefore possible to further prevent the electrode part or the wiring part from being exposed.

In the living body-attachable electrode according to an aspect of the disclosure, the adhesive layer includes, for example, an adhesive resin such as an acrylic polymer or a urethane resin.

In the living body-attachable electrode according to an aspect of the disclosure, a thickness of the adhesive layer is not limited, but is desirably 10 μm or less, and more desirably 1 μm or less. The thickness of the adhesive layer is desirably 0.1 μm or more.

The living body-attachable electrode according to an aspect of the disclosure does not have to include an adhesive layer.

FIG. 11 is a sectional view schematically showing a living body-attachable electrode according to an aspect of the disclosure.

A living body-attachable electrode 5 shown in FIG. 11 has the same configuration as the living body-attachable electrode 3 shown in FIG. 9 except that a recess 30X is provided on the main surface of the second stretchable substrate 30 not in contact with the first stretchable substrate 10, and that the gel electrode 40 is embedded in the recess 30X. The living body-attachable electrode 3 shown in FIG. 9, the living body-attachable electrode 1 shown in FIG. 1, the living body-attachable electrode 2 shown in FIG. 6, and the living body-attachable electrode 4 shown in FIG. 10, the recess 30X may be provided on the main surface of the second stretchable substrate 30 not in contact with the first stretchable substrate 10, and the gel electrode 40 may be embedded in the recess 30X.

In the living body-attachable electrode according to an aspect of the disclosure, as shown in FIG. 11, the recess is desirably provided on the main surface of the second stretchable substrate not in contact with the first stretchable substrate, and that the gel electrode is desirably embedded in the recess.

The gel electrode, which is embedded in the recess provided on the main surface of the second stretchable substrate, makes the gel electrode less likely to peel off due to the anchor effect.

In the living body-attachable electrode according to an aspect of the disclosure, a width of the recess is not limited, but is desirably 1 μm or more and 100 μm or less. A depth of the recess is not limited, but is desirably 1% or more and 75% or less of the thickness of the second stretchable substrate, and is desirably 1 μm or more and 30 μm or less, for example.

In the living body-attachable electrode according to an aspect of the disclosure, the gel electrode, embedded in the recess, may be embedded in the entire recess, or may be embedded in a part of the recess.

Hereinafter, a method of manufacturing the living body-attachable electrode according to an aspect of the disclosure will be described.

As an example of the method of manufacturing the living body-attachable electrode of according to an aspect of the disclosure, a method of manufacturing the living body-attachable electrode 1 shown in FIG. 1 will be described.

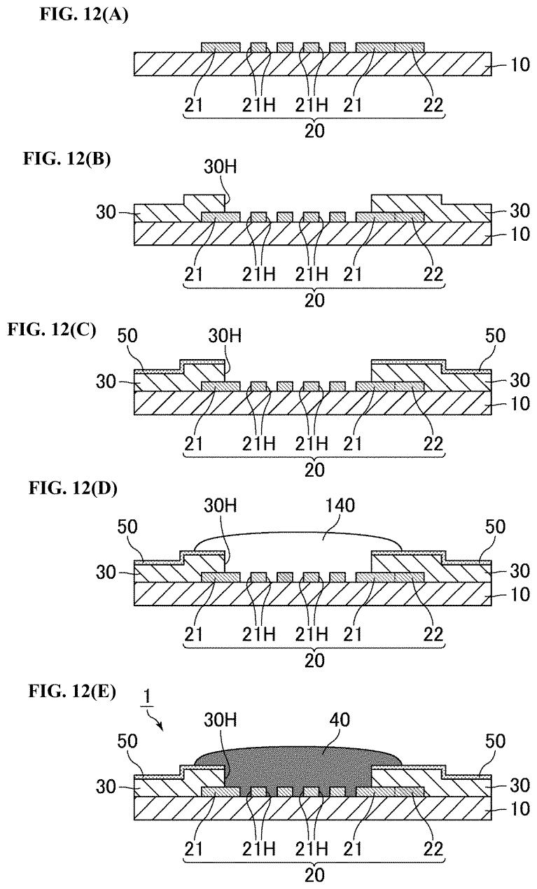

FIGS. 12 (A)-(E) are sectional views schematically showing a method of manufacturing the living body-attachable electrode according to an aspect of the disclosure. FIGS. 13A, 13B, 13C, 13D, and 13E are top views schematically showing the method of manufacturing the living body-attachable electrode according to an aspect of the disclosure.

First, as shown in FIGS. 12A and 13A, the conductive pattern 20 including the electrode part 21 and the wiring part 22 connected to the electrode part 21 is formed on the one main surface of the first stretchable substrate 10. In FIGS. 12A and 13A, the electrode part 21 forms a conductive pattern 20 having through holes 21H.

For example, the conductive pattern is formed by printing a conductive paste on the one main surface of the first stretchable substrate in a predetermined pattern.

The electrode part and the wiring part are desirably formed by an identical process using the identical conductive paste, but may be formed by separate processes using the identical or different conductive pastes.

Next, as shown in FIGS. 12B and 13B, the second stretchable substrate 30 having the opening 30H is disposed on the one main surface of the first stretchable substrate 10. In this step, at least the wiring part 22 of the conductive pattern 20 is covered with the second stretchable substrate 30, and at least a part of the electrode part 21 is exposed from the opening 30H of the second stretchable substrate 30. In FIGS. 12B and 13B, in addition to the wiring part 22, the entire periphery of the electrode part 21 is also covered with the second stretchable substrate 30.

For example, the second stretchable substrate is attached to the first stretchable substrate by bonding the first stretchable substrate and the second stretchable substrate to each other by thermal pressure.

Upon manufacturing the living body-attachable electrode 1 shown in FIG. 1, as shown in FIGS. 12C and 13C, the adhesive layer 50 is formed on the main surface of the second stretchable substrate 30 not in contact with the first stretchable substrate 10.

For example, an adhesive resin is applied to the main surface of the second stretchable substrate, and then dried and/or cured to form the adhesive layer.

Instead of forming the adhesive layer after placing the second stretchable substrate on the one main surface of the first stretchable substrate, the second stretchable substrate on which the adhesive layer is formed may be disposed on the one main surface of the first stretchable substrate.

Subsequently, as shown in FIGS. 12D and 13D, a gel electrode material 140 is applied inside the opening 30H of the second stretchable substrate 30.

When the gel electrode material is applied inside the opening of the second stretchable substrate, the gel electrode material protrudes from the opening, and adheres to the main surface of the second stretchable substrate, the main surface being not in contact with the first stretchable substrate. Further, the gel electrode material in the opening protrudes from the second stretchable substrate in the thickness direction due to surface tension. Specifically, the gel electrode material has a protruding shape being thicker from the peripheral edge toward the center.

Examples of a method of applying the gel electrode material include a dispensing method and an inkjet method. The gel electrode material is desirably applied by the dispense method.

Then, as shown in FIGS. 12E and 13E, the gel electrode material 140 is cured to form the gel electrode 40 conductive to the electrode part 21.

Examples of a method of curing the gel electrode material include ultraviolet curing (UV curing) and the like.

From the above, the living body-attachable electrode 1 shown in FIG. 1 is obtained. In the living body-attachable electrode 1 thus obtained, the gel electrode 40 is formed within the opening 30H of the second stretchable substrate 30 and also on the main surface of the second stretchable substrate 30 not in contact with the first stretchable substrate 10 so as to spread outward from the opening 30H of the second stretchable substrate 30.

In the above manufacturing method, the conductive pattern 20 including the electrode part 21 having no through holes 21H is formed, and then the living body-attachable electrode 2 shown in FIG. 6 is obtained.

In the above manufacturing method, the part of the periphery of the electrode part 21 that is unconnected to the wiring part 22 is not covered with the second stretchable substrate 30, and then the living body-attachable electrode 3 shown in FIG. 9 is obtained.

In the above manufacturing method, the adhesive layer is formed after the gel electrode is formed, and then the living body-attachable electrode 4 shown in FIG. 10 is obtained.

Upon manufacturing the living body-attachable electrode 3 shown in FIG. 9, the gel electrode material 140 is applied after the recess 30X is formed on the main surface of the second stretchable substrate 30 not in contact with the first stretchable substrate 10, and then the living body-attachable electrode 5 shown in FIG. 11 is obtained.

In the method of manufacturing the living body-attachable electrode according to an aspect of the disclosure, the adhesive layer does not have to be formed.

In the method of manufacturing the living body-attachable electrode according to an aspect of the disclosure, the gel electrode having high stretchability can be formed compared with a method of forming the gel electrode by placing a gel sheet having a predetermined shape in the opening of the second stretchable substrate. Therefore, the living body-attachable electrode whose stretchability of the gel electrode is identical to or higher than that of the first and second stretchable substrates can be manufactured.

The living body-attachable electrode according to an aspect of the disclosure and the method of manufacturing the living body-attachable electrode are not limited to the above disclosure. Various applications and modifications can be made to the configuration, the manufacturing conditions, and the like of the living body-attachable electrode within the scope of the disclosure.

For example, a configuration of the conductive pattern (shape, number, arrangement, and the like of the electrode part) can be appropriately changed in accordance with the use or purpose of the living body-attachable electrode. When there is a plurality of the electrode parts, the gel electrode desirably extends to the outside of the opening of the second stretchable substrate for every electrode part. However, there may be an electrode part in which the gel electrode does not extend to the outside of the opening of the second stretchable substrate.

Further, the material, size, and the like of the first stretchable substrate, the second stretchable substrate, the gel electrode, and the like are mere examples, and are not limited to those disclosed in the above embodiments.

The description of the aspects disclosed should be considered as being illustrative in all respects and not being restrictive. The scope of the present invention is shown by the claims rather than by the above description, and is intended to include meanings equivalent to the claims and all changes in the scope. While preferred aspects of the invention have been described above, it is to be understood that variations and modifications will be apparent to those skilled in the art without departing from the scope and spirit of the invention.

### DESCRIPTION OF REFERENCE SYMBOLS

1, 2, 3, 4, 5: Living body-attachable electrode
10: First stretchable substrate
20: Conductive pattern
21: Electrode part
21H: Through hole
22: Wiring part
30: Second stretchable substrate
30H: Opening
30X: Recess
40: Gel electrode
50: Adhesive layer
140: Gel electrode material
HB: Human body

What is claimed is:

1. A living body-attachable electrode comprising:
a first stretchable substrate;
a conductive pattern on a surface of the first stretchable substrate and including an electrode and a wiring connected to the electrode;
a second stretchable substrate on the surface of the first stretchable substrate and configured to cover the wiring of the conductive pattern and including an opening exposing a portion of the electrode; and
a gel electrode in the opening of the second stretchable substrate and conductive to the electrode, the gel electrode spreading outward from the opening of the second stretchable substrate onto a first surface of the second stretchable substrate.

2. The living body-attachable electrode according to claim 1, wherein the gel electrode protrudes in a thickness direction from the second stretchable substrate.

3. The living body-attachable electrode according to claim 2, wherein the gel electrode includes a protruding shape that is thicker in a direction from a peripheral edge toward a center of the gel electrode.

4. The living body-attachable electrode according to claim 1, wherein the electrode includes a plurality of recesses recessed in a thickness direction from a top surface thereof that is not in contact with the first stretchable substrate.

5. The living body-attachable electrode according to claim 4, wherein the plurality of recesses include through holes penetrating the electrode in the thickness direction.

6. The living body-attachable electrode according to claim 5, wherein, when the through holes of the electrode have a radius r and a thickness t, r≤2t.

7. The living body-attachable electrode according to claim 1, wherein the second stretchable substrate includes a recess on the first surface of the second stretchable substrate, and the gel electrode is embedded in the recess, the first surface of the second stretchable substrate not being in contact with the first stretchable substrate.

8. The living body-attachable electrode according to claim 1, further comprising an adhesive layer on the first surface of the second stretchable substrate, the first surface not being in contact with the first stretchable substrate.

9. The living body-attachable electrode according to claim 8, wherein the adhesive layer is between the first surface of the second stretchable substrate and the gel electrode.

10. The living body-attachable electrode according to claim 1, wherein the gel electrode in the opening of the second stretchable substrate has a non-uniform cross-sectional shape.

11. A method of manufacturing a living body-attachable electrode comprising:
forming a conductive pattern including an electrode and a wiring connected to the electrode on a surface of a first stretchable substrate;
placing a second stretchable substrate having an opening on the surface of the first stretchable substrate so as to cover the wiring of the conductive pattern with the second stretchable substrate, and expose a portion of the electrode from the opening of the second stretchable substrate;
applying a gel electrode material conductive to the electrode inside the opening of the second stretchable substrate and on a first surface of the second stretchable substrate by curing the gel electrode material.

12. The method according to claim 11, wherein the gel electrode provided in the opening of the second stretchable substrate protrudes in a thickness direction from the second stretchable substrate.

13. The method according to claim 11, wherein the gel electrode includes a protruding shape that is thicker in a direction from a peripheral edge toward a center of the gel electrode.

14. The method according to claim 11, wherein the electrode includes a plurality of recesses recessed in a thickness direction from a top surface thereof that is not in contact with the first stretchable substrate.

15. The method according to claim 14, wherein the plurality of recesses include through holes penetrating the electrode in the thickness direction.

16. The method according to claim 15, wherein, when the through holes of the electrode have a radius r and a thickness t, r≤2t.

17. The method according to claim 11, wherein the second stretchable substrate includes a recess on the first surface of the second stretchable substrate, and the gel electrode is embedded in the recess, the first surface not being in contact with the first stretchable substrate.

18. The living body-attachable electrode according to claim 11, further comprising applying an adhesive layer on the first surface of the second stretchable substrate, the first surface not being in contact with the first stretchable substrate.

19. The living body-attachable electrode according to claim 18, wherein the adhesive layer is provided between the first surface of the second stretchable substrate and the gel electrode.

20. The method according to claim 11, wherein the gel electrode provided in the opening of the second stretchable substrate has a non-uniform cross-sectional shape.
